(19) United States
(12) Patent Application Publication
(10) Pub. No.: US 2002/0137076 A1
(43) Pub. Date: Sep. 26, 2002

(54) RNA polymers and uses thereof

(76) Inventors: John W. Shultz, Verona, WI (US); Martin K. Lewis, Madison, WI (US); Christine Andrews, Cottage Grove, WI (US)

Correspondence Address: MEDLEN & CARROLL, LLP, 101 HOWARD STREET, SUITE 350, SAN FRANCISCO, CA, 94105, US

(21) Appl. No.: 09/995,912

(22) Filed: Nov. 28, 2001

Related U.S. Application Data
(60) Provisional application No. 60/253,451, filed on Nov. 28, 2000.

(51) Int. Cl. C12Q 1/68; C12N 9/99; C12M 1/34

(52) U.S. Cl. 435/6; 435/184; 435/287.2

(57) ABSTRACT

The present invention relates to compositions and methods for altering enzyme reactions. In particular, the invention relates to composition and methods for using RNA polymers to inhibit RNase enzymes, to remove RNA-binding enzymes and proteins from solution and to enhance certain enzymatic reactions.

The present invention claims priority to U.S. Provisional Patent Appl. No. 60/253,451, filed Nov. 28, 2000, the disclosure of which is hereby incorporated by reference in its entirety.

### FIELD OF THE INVENTION

The present invention relates to compositions and methods for altering enzyme reactions. In particular, the present invention provides compositions and methods for using RNA polymers to inhibit RNase enzymes, to remove RNA binding proteins from solution, and to enhance certain enzymatic reactions.

### BACKGROUND OF THE INVENTION

Ribonucleases (RNases) are enzymes that degrade ribonucleic acid (RNA). RNases are used in vitro to remove unwanted RNA from molecular biology procedures. Some RNase enzymes preferentially cleave at specific sequences, for example, after particular ribonucleotides, and are used in vitro for RNA sequencing. However, the common use of RNase enzymes and the presence of RNase enzymes on the skin of laboratory personnel often results in unwanted contamination of laboratory reactions with RNase enzymes. Such contamination is detrimental to reactions utilizing RNA substrates or generating RNA molecules. Other RNA binding proteins used in molecular biology procedures (e.g., reverse transcriptase enzymes) also can become unwanted contaminants in subsequent steps of a multi-step procedure.

In vivo, RNases serve as cytotoxic agents in host cell defense against viral infection and in physiological cell death pathways in bacteria, higher plants, and mammals. The RNA population in cells is controlled post-transcriptionally by RNases of varying specificity. RNase activity in serum and cell extracts is elevated in a variety of cancers and infectious diseases. RNases are regulated by specific activators and inhibitors, including interferon. Many of these regulatory molecules, in addition to the RNase molecules, are targeted in drug design for drugs to control tumor angiogenesis, allergic reactions, and viral infections (Schein, Nat. Biotechnol., 15:529 [1997]).

Human angiogenin is a member of the pancreatic RNase superfamily and is a potent inducer of angiogenesis, the development of new blood vessels, in vivo. In vitro, angiogenin induces cell migration and formation of tube-like structures by endothelial cells on collagen gel. Angiogenin catalyzes limited cleavage of 28S and 18S rRNAs and transfer RNAs and produces fragments 100-500 nucleotides in length. Placental ribonuclease inhibitor prevents angiogenin from binding to calf pulmonary artery endothelial cells in culture (Badet et al., PNAS, 86:8427 [1989]). Increased angiogenin levels have been implicated in the establishment of a wide variety of tumors, including renal cell carcinoma, colorectal cancer, malignant melanoma, urothelial carcinoma, a variety of brain tumors, gastric cancer, and epithelial ovarian cancer (See e.g., Wechsel et al., Anticancer Res., 19:1537 [1999]; Montero et al., Clin Caner Res., 5:3722 [1999]; Hartmann et al., Cancer Res., 59:1578 [1999]; Miyake et al, Cancer, 86:316 [1999]; Eberle et al., Anticancer Res., 20:1679 [2000]; Shimoyama and Kaminishi, J. Cancer Res. Clin. Oncol., 126:468 [2000]; Barton et al., Clin. Cancer Res., 3:1579 [1997]). Consequently, many anti-cancer therapies have targeted angiogenin as a method of inhibiting tumor growth.

Anti-angiogenin cancer therapies have generally focused on protein or peptide therapies. For example, anti-angiogenin peptides have been generated and shown to inhibit angiogenesis in vivo and in vitro (See e.g., Gho et al., Cancer Res., 57:3733 [1997]; Gho and Chae, J. Biol. Chem., 272:24294 [1997]). Other anti-cancer therapies that target angiogenin utilize monoclonal antibodies against angiogenin. Monoclonal mouse antibodies against angiogenin have been shown to be effective in interfering with the spread of tumors in mice (Piccoli et al., PNAS, 95:4579 [1998]; Olson et al., PNAS, 92:442 [1995]).

However, such antibodies are not likely to be plausible treatments for human tumors because of immune responses against the mouse antibodies. Chimeric mouse-human antibodies have been generated in an attempt to eliminate the immune response (Piccoli et al., PNAS, 95:4579 [1998]). However, peptides and antibodies are time-consuming and expensive to produce on a commercial scale.

The art is in need of easy to produce, cost-effective methods and compositions for inhibiting RNase enzymes in living cells and in vitro and for removing RNA binding proteins from solution, thereby enhancing certain enzymatic reactions.

### DESCRIPTION OF THE FIGURE

FIG. 1 shows the Lineweaver-Burke plot of polymer polyG inhibition of RNase A

### SUMMARY OF THE INVENTION

The present invention relates to compositions and methods for altering enzyme reactions. In particular, the present invention relates to compositions and methods for using RNA polymers to inhibit RNase enzymes, to remove RNA binding proteins (e.g., enzymes) from solution, and to enhance certain enzymatic reactions.

The present invention provides a method, comprising: providing a preparation comprising at least one RNA polymer (e.g., tRNA, RNA heteropolymers, RNA homopolymers, etc.); a sample containing an RNA binding enzyme; and mixing the preparation with the sample under conditions such that the activity of the RNA binding enzyme is diminished at least 25% relative to the activity of the RNA binding enzyme in the absence of said RNA polymer. In some embodiments, the activity of the RNA binding enzyme is diminished at least 50% relative to the activity of the RNA binding enzyme in the absence of the RNA polymer. In other embodiments, the activity of the RNA binding enzyme is diminished at least 75% relative to the activity of the RNA binding enzyme in the absence of the RNA polymer. In still further embodiments, the activity of the RNA binding enzyme is diminished at least 90% relative to the activity of the RNA binding enzyme in the absence of the RNA polymer.

The present invention is not limited to any one RNA polymer. Indeed, a variety of RNA polymers are contemplated including, but not limited to, polyA:polyU; polyC:polyG; polyC:polyI; polyI; polyC; polyA; polyG; poly(GU); poly(CU); poly(GI) and poly(CI). It is also contemplated that tRNA molecules find use in the methods of the present invention. In some embodiments, the RNA polymers are affixed to a solid support. In some embodiments, the solid support is a resin. In other embodiments, the solid support is a plastic tube or plate.

In some embodiments, the RNA-binding enzyme comprises an RNAse. The present invention is not limited to any one RNase. Indeed, a variety of RNase enzymes are contemplated including, but not limited to, RNase A, RNase H, RNase One, RNase B, RNase T₁, RNase T₂, RNase S, RNase from chicken liver, and RNase from Aspergillus clavatus. In some embodiments, the preparation further comprises a ribonuclease inhibitor.

In some embodiments, the RNA-binding enzyme is in a cell. In some embodiments, the cell is a tumor cell. In some embodiments, the RNA-binding enzyme comprises angiogenin.

The present invention also provides a method, comprising: providing a preparation comprising at least one RNA polymer; a sample containing an RNase enzyme; and mixing the preparation with the sample under conditions such that the activity of the RNase enzyme is diminished at least 25% relative to the activity of the RNase enzyme in the absence of the RNA polymer. In some embodiments, the activity of the RNase enzyme is diminished at least 50% relative to the activity of the RNase enzyme in the absence of the RNA polymer. In other embodiments, the activity of the RNase enzyme is diminished at least 75% relative to the activity of the RNase enzyme in the absence of the RNA polymer. In still further embodiments, the activity of the RNase enzyme is diminished at least 90% relative to the activity of the RNase enzyme in the absence of said RNA polymer.

The present invention is not limited to any one RNA polymer. Indeed, a variety of RNA polymers are contemplated including, but not limited to, polyA:polyU; polyC:polyG; polyC:polyI; polyI; polyC; polyA; polyG; poly(GU); poly(CU); poly(GI) and poly(CI). In some embodiments, the RNA polymers are affixed to a solid support. In some embodiments, the solid support is a resin. In other embodiments the solid support is a plastic tube or plate.

In some embodiments, the RNA-binding enzyme comprises an RNAse. The present invention is not limited to any one RNase. Indeed, a variety of RNase enzymes are contemplated including, but not limited to, RNase A, RNase H, RNase One, RNase B, RNase T₁, RNase T₂, RNase S, RNase from chicken liver, and RNase from Aspergillus clavatus. In some embodiments, the preparation further comprises a ribonuclease inhibitor. In some embodiments, the RNase inhibitor is RNASIN.

In some embodiments, the RNase enzyme is part of a cell. In some embodiments, the cell is a tumor cell. In some embodiments, the RNase enzyme comprises angiogenin.

The present invention further provides a method for selling an RNase inhibitor, comprising: providing a kit comprising at least one RNA polymer; and providing the kit to a customer. The present invention is not limited to any one RNA polymer. Indeed, a variety of RNA polymers are contemplated including, but not limited to, polyA:polyU; polyC:polyG; polyC:polyI; polyI; polyC; polyA; polyG; poly(GU); poly(CU); poly(GI) and poly(CI). In some embodiments, the RNA polymers are affixed to a solid support. In some embodiments, the kit further comprises RNASIN RNase inhibitor.

In some embodiments, the kit further comprises a delivery system. In some embodiments, the delivery system comprises a solid support. In some embodiments, the solid support is a resin. In some embodiments, the RNA polymers are affixed to said solid support. In other embodiments, the delivery system comprises a plastic reaction vessel (e.g., a tube or a plate). In some embodiments, the RNA polymers are affixed to the plastic reaction vessel. In some embodiments, prior to providing the kit to the customer, the RNA polymer is tested in an RNase inhibition assay.

The present invention additionally provides a system, comprising: at least one RNA polymer capable of inhibiting the activity of a RNA binding enzyme at least 25% relative to the activity of said RNA binding enzyme in the absence of said RNA polymer; and a delivery means. In some embodiments, the RNA polymer is capable of inhibiting the activity of a RNA binding enzyme at least 50% relative to the activity of the RNA binding enzyme in the absence of the RNA polymer. In other embodiments, the RNA polymer is capable of inhibiting the activity of a RNA binding enzyme at least 75% relative to the activity of the RNA binding enzyme in the absence of the RNA polymer. In still further embodiments, the RNA polymer is capable of inhibiting the activity of a RNA binding enzyme at least 90% relative to the activity of the RNA binding enzyme in the absence of the RNA polymer.

In some embodiments, the system further comprises a RNase inhibitor. In some embodiments, the RNase inhibitor is RNASIN. In some embodiments, the delivery system comprises a solid support. In some embodiments, the solid support is a resin. In some embodiments, the RNA polymers are affixed to the solid support. In other embodiments, the delivery system comprises a plastic reaction vessel. In some embodiments, the RNA polymers are affixed to the plastic reaction vessel.

In some embodiments, the system further comprises components necessary for performing an enzymatic reaction. In some embodiments, the enzymatic reaction is one-step reverse-transcription PCR. In other embodiments, the enzymatic reaction is two-step reverse-transcription PCR. In some embodiments, the reverse-transcription PCR reaction components comprise a reverse transcription enzyme selected from the group consisting of avian myeloblastosis virus reverse transcriptase enzyme and moloney murine leukemia reverse transcriptase enzyme.

The present invention also provides a method for enhancing an enzymatic reaction, comprising: providing a preparation comprising at least one RNA polymer; reaction components necessary for a reverse-transcription PCR reaction; and mixing the preparation with said reaction components under conditions such that the level of detectable reaction product is increased relative to the level in the absence of said RNA polymer. In some embodiments, the reverse-transcription PCR reaction is one-step reverse-transcription PCR. In other embodiments, the reverse-transcription PCR reaction is a two-step reverse-transcription PCR. In some embodiments, the reverse-transcription PCR reaction components comprise a reverse transcription enzyme selected from the group consisting of avian myeloblastosis virus reverse transcriptase enzyme and moloney murine leukemia reverse transcriptase enzyme. The present invention is not limited to any one RNA polymer. Indeed, a variety of RNA polymers are contemplated including, but not limited to, polyA:polyU; polyC:polyG; polyC:polyI; polyI; polyC; polyA; polyG; poly(GU); poly(CU); poly(GI) and poly(CI).

The present invention further provides a method, comprising: providing a preparation comprising at least one RNA polymer; a first sample comprising an RNA binding enzyme; a second sample comprising an RNA molecule of interest; and mixing the preparation with the first sample and the second sample under conditions such that the activity of the RNA binding enzyme is diminished at least 25% relative to the activity of said RNA binding enzyme in the absence of said RNA polymer. In some embodiments, the activity of the RNA binding enzyme is diminished at least 50% relative to the activity of the RNA binding enzyme in the absence of the RNA polymer. In other embodiments, the activity of the RNA binding enzyme is diminished at least 75% relative to the activity of the RNA binding enzyme in the absence of the RNA polymer. In still further embodiments, the activity of the RNA binding enzyme is diminished at least 90% relative to the activity of the RNA binding enzyme in the absence of the RNA polymer.

In some embodiments, the preparation is mixed with the first sample prior to mixing with the second sample. The present invention is not limited to any one RNA polymer. Indeed, a variety of RNA polymers are contemplated including, but not limited to, polyA:polyU; polyC:polyG; polyC:polyI; polyI; polyC; polyA; polyG; poly(GU); poly(CU); poly(GI) and poly(CI). In some embodiments, the RNA polymers are affixed to a solid support. In some embodiments, the RNA binding enzyme comprises an RNAse. In some embodiments, the RNAse is selected from the group consisting of: RNase A, RNase H, RNase One, RNase B, RNase T₁, RNase T₂, RNase S, RNase from chicken liver, and RNase from Aspergillus clavatus.

The present invention additionally provides a method of removing a RNA binding enzyme from a sample, comprising: providing a preparation comprising at least one RNA polymer; a sample comprising an RNA binding enzyme; and mixing the preparation with the sample under conditions such that at least 25% of the RNA binding enzyme is bound by the RNA polymer. In some embodiments, at least 50% of the RNA binding enzyme is bound by the RNA polymer. In other embodiments, at least 75% of the RNA binding enzyme is bound by the RNA polymer. In still further embodiments, at least 90% of the RNA binding enzyme is bound by the RNA polymer.

The present invention is not limited to any one RNA polymer. Indeed, a variety of RNA polymers are contemplated including, but not limited to, polyA:polyU; polyC:polyG; polyC:polyI; polyI; polyC; polyA; polyG; poly(GU); poly(CU); poly(GI) and poly(CI). In some embodiments, the RNA polymers are affixed to a solid support. In some embodiments, the RNA binding enzyme comprises an RNAse. In some embodiments, the RNAse is selected from the group consisting of: RNase A, RNase H, RNase One, RNase B, RNase T₁, RNase T₂, RNase S, RNase from chicken liver, and RNase from Aspergillus clavatus.

### DEFINITIONS

To facilitate understanding of the invention, a number of terms are defined below.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “oligonucleotide” as used herein is defined as a molecule comprised of two or more deoxyribonucleotides or ribonucleotides, preferably more than three, and usually ten or more. The exact size will depend on many factors, which in turn depends on the ultimate function or use of the oligonucleotide. The oligonucleotide may be generated in any manner, including, but not limited to, chemical synthesis, DNA replication, reverse transcription, or a combination thereof.

As used herein, the term “ssRNA” refers to a single-stranded RNA molecule or oligoribonucleotide. A “ssRNA” may be a homopolymer (e.g., polyI, polyC or polyG) or may be a heterogeneous polymer composed of, for example, a random sequence of different ribonucleotides or simply two different ribonucleotides. In addition, ssRNA may comprise regions of secondary or tertiary structure.

As used herein, the term “dsRNA” refers to two complementary RNA molecules that have annealed to one-another to form a double stranded RNA molecule. The two strands may be complementary homopolymers of RNA (e.g., a “polyI:polyC” dsRNA molecule) or may alternatively be random complementary sequences (e.g., the “sense” and “antisense” RNAs of a “gene”).

As used herein the terms, “polyI,” “polyC,” “polyG,” and the like, refer to ribonucleic acid homopolymers (e.g., polyinosine, polycytosine, polyguanosine, and the like). “Homopolymers” are nucleic acid polymers having a single type of nucleotide (e.g., A, G, C, T, U, I). As used herein, the term “poly(CU)” and the like, refers to ssRNA polymers comprised of a heterogeneous sequence of the ribonucleotides named in the parentheses. “Heteropolymers” are nucleic acid polymers having at least two different nucleotide constituents. In some embodiments, heteropolymers comprise a single stranded nucleic acid with at least two different nucleotides (e.g., poly(CU)). In some embodiments, heteropolymers comprise double strand nucleic acid wherein the individual strands of the heteropolymer are either homopolymers or heteropolymers.

As used herein, the term “RNA polymer” refers to an RNA molecule of two or more ribonucleotides, preferably more than three, and usually ten or more. RNA polymers may be ssRNA or dsRNA, natural or synthetic.

As used herein, the term “RNA molecule of interest” refers to any RNA molecule for which protection from degradation (e.g., by RNA polymers of the present invention) is desired. Examples of “RNA molecules of interest” include those produced or utilized by enzymatic reactions (e.g., reverse transcription templates or the products of in vitro transcription).

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.”Complementarity may be “partial,” in which only some of the nucleic acid bases are matched according to the base pairing rules. Or, there may be “complete” or “total” complementarity between the nucleic acids. The degree of complementarity between nucleic acid strands has significant effects on the efficiency and strength of hybridization between nucleic acid strands.

This is of particular importance in amplification reactions, as well as detection methods that depend upon binding between nucleic acids.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature” of a nucleic acid. The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value for DNA:DNA hybrids may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). DNA:RNA and RNA:RNA hybrids will have a higher Tₘ than the equivalent DNA:DNA hybrid (See e.g., Wetmur, Crit. Rev. Biochem. and Mol. Biol. 26:227-[1991]).

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The lengths of the primers will depend on many factors, including temperature, source of primer and the use of the method.

As used herein, the term “polymerase chain reaction” (“PCR”) refers to the method of K. B. Mullis; U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “reverse-transcription PCR” or “RT-PCR” refers to a type of PCR where the starting material is RNA. The starting RNA is enzymatically converted to complementary DNA or “cDNA” using a reverse transcriptase enzyme. The cDNA is then used as a “template” for a “PCR” reaction.

As used herein, the term “antisense” is used in reference to nucleic acid sequences that are complementary to a specific RNA sequence (e.g., mRNA). The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a polypeptide of interest includes, by way of example, such nucleic acid in cells ordinarily expressing the polypeptide where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” or when in reference to a nucleotide sequence (as in “a portion of a given nucleotide sequence”) refers to segments of that sequence. The segments may range in size from four nucleotides to the entire nucleotide sequence minus one nucleotide (e.g., 4, 5, 6, 7, . . . , n-1).

As used herein, the term “host cell” refers to any eukaryotic cell or prokaryotic organism (e.g., bacteria, single-celled eukaryotic organisms (e.g., yeast or protozoa), mammalian cells, avian cells, amphibian cells, plant cells, fish cells, and insect cells), whether located in vitro, in situ or in vivo.

As used herein, the term “cell culture” refers to any in situ culture of cells. Included within this term are continuous cell lines (e.g., with an immortal phenotype), primary cell cultures, finite cell lines (e.g., non-transformed cells), and any other cell population maintained in situ, including, but not limited to yeast, bacterial, plant, mammalian, and insect cells.

As used herein, the terms “restriction endonucleases” and “restriction enzymes” refer to bacterial enzymes, each of which cut double-stranded DNA at or near a specific nucleotide sequence.

As used herein, the terms “RNA binding enzyme” or “RNA binding protein” refer to any enzyme or protein capable of binding to RNA. One example of a class of “RNA binding enzymes” are the “RNases.” As used herein, the term “RNase,” or its equivalent “RNases” refers to enzymes that hydrolyze RNA to produce smaller RNA fragments or ribonucleotides.

As used herein the term “reduces the activity,” when used in reference to a compound (e.g., an RNA polymer of the present invention) refers to a compound that reduces one or more activities (e.g., RNA binding or RNase activity) of a given enzyme or protein relative to the activity in the absence of the compound. Preferred compound are those that decrease the activity by at least 25%. More preferred compounds are those that decrease the activity by at least 50%. Even more preferred compounds are those that decrease the activity by at least 75%, and more preferably, 90%. The activity of a given enzyme or protein may be measured using any suitable method including but not limited to, those disclosed herein. For Example, assays for measuring the activity of an enzyme or protein include, but are not limited to, those described in Examples 8-12.

As used herein, the term “inhibits the activity,” when used in reference to a compound (e.g., an RNA polymer of the present invention) refers to a compound that “reduces the activity” of a given enzyme or protein.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the term “purified” or “to purify” means the result of any process which removes some contaminants from the component of interest, such as a protein or nucleic acid. The percent of a purified component is thereby increased in the sample.

As used herein the term “portion” when used in reference to a protein (as in “a portion of a given protein”) refers to fragments of that protein. The fragments may range in size from four amino acid residues to the entire amino acid sequence minus one amino acid (e.g., 4, 5, 6, 7, . . . , n-1).

The term “test compound” refers to any chemical entity, pharmaceutical, drug, and the like that can be used to treat or prevent a disease or infection, or otherwise alter the physiological or cellular status of a sample (e.g., a cell or organism). Test compounds comprise both known and potential therapeutic compounds. A test compound can be determined to be therapeutic by screening using the screening methods of the present invention. A “known therapeutic compound” refers to a therapeutic compound that has been shown (e.g., through administration to a subject) to be effective in such treatment or prevention. In other words, a known therapeutic compound is not limited to a compound efficacious in the treatment of pathological conditions such as disease or viral infection.

As used herein, the term “sample” is used in its broadest sense. In one sense it can refer to a tissue sample. In another sense, it is meant to include a specimen or culture obtained from any source, as well as biological. Biological samples may be obtained from animals or plants and encompass fluids, solids, tissues, and gases. Biological samples include, but are not limited to tissues, cells, or extracts. These examples are not to be construed as limiting the sample types applicable to the present invention.

### DETAILED DESCRIPTION OF THE INVENTION

In some embodiments, the present invention provides methods of inhibiting RNase enzymes (e.g., in enzymatic reactions). In other embodiments, the present invention provides methods of removing contaminating RNA binding enzymes (e.g., RNase enzymes) from a reaction mixture. In still other embodiments, the present invention provides methods of enhancing enzymatic reactions. In yet other embodiments, the present invention provides methods of inhibiting cellular RNases involved in angiogenesis (e.g., tumor growth and proliferation) and other biological processes. Each of these embodiments is described below in greater detail. The description below provides specific, but not limiting, illustrative examples of uses of RNA polymers in the methods described herein.

RNases are common contaminants of laboratory reactions. RNaseA is present on human skin and is often found on laboratory equipment and supplies. RNases are often added to common laboratory practices, such as plasmid purification, protein purification, and certain molecular biology assays such as ribonuclease protection assays; therefore, residual RNase enzyme may contaminate solutions and equipment. Further, RNases are often found as contaminants in experiments that utilize bacterial lysates, as well as in proteins or DNA purified from bacteria. Thus, RNases have the potential to contaminate and disrupt any molecular biological or biochemical assay that utilizes or generates RNA. RNase ONE is the major RNase of E. coli and is a very active enzyme with very little base specificity. RNase ONE ribonuclease will not cleave RNA-DNA hybrid molecules, but will cleave single-stranded RNA. As such the enzyme is useful in RNase protection assays.

Commercial RNase inhibitors (e.g., RNASIN (Promega Corp), SUPERASEIN (Ambion); and RNAGUARD (Amersham Pharmacia Biotech)) are generally proteins that have a limited range of specificity and are often sensitive to solution conditions (e.g., pH) and inactivation by heat. The present invention provides novel compositions and methods of inhibiting RNase enzymes that overcome many of the limitations of existing inhibitors. The RNA polymers of the present invention inhibit a wide range of RNase enzymes and are not inactivated by elevated temperatures used in many enzymatic reactions. The RNA polymers have the added advantage that they precipitate along with the RNA of interest (e.g. in an RNase Protection Assay), thereby acting both as a carrier for quantitative precipitation of the sample and as an inhibitor of RNase enzymes which would follow the sample, protecting it until it is separated from the RNA polymer (e.g. during electrophoresis).

I. Methods of Inhibiting RNA Binding Enzymes

In some embodiments, the present invention provides methods of inhibiting the activity of RNA binding enzymes (e.g., including, but not limited to, RNase enzymes, polyA polymerases, reverse transcriptases, and small nuclear ribonucleoproteins). In some embodiments, a suitable RNA polymer is employed to inhibit RNAse enzymes, including, but not limited to double stranded polyA:polyU; polyC:polyG and polyC:polyI; and single stranded polyI; polyC; polyG; polyA; poly(GU); poly(CU); poly(GI) and poly(CI). In other embodiments, other suitable RNA polymers are utilized. In some embodiments, two or more RNA polymers are combined. In some embodiments, additional RNA inhibitors (e.g., RNASIN Ribonuclease Inhibitor (Promega, Madison, Wis.)) are combined with a RNA polymer. The methods of the present invention are used to inhibit RNase enzymes in a reaction or to remove RNA binding enzymes (e.g., RNases) from a solution.

A. Suitable RNA Polymers

In some embodiments of the present invention, double-stranded RNA (dsRNA) polymers (e.g., polyI:polyC or polyA:polyU or HeLa RNA) are utilized to inhibit RNAse enzymes. In other embodiments, ssRNA polymers (e.g., polyG; polyI, polyA, polyC, poly(GU), poly(CU), poly(GI), poly(CI), or a mixture thereof) are utilized to inhibit RNAse enzymes. It is also contemplated that tRNA molecules find use in the methods of the present invention.

The RNA polymers of the present invention are not limited to any particular length of polymer (i.e., number of ribonucleotides). Indeed, a variety of lengths are contemplated. Accordingly, in some preferred embodiments, the RNA polymers are very long (e.g., molecular weights of 100,000 to 1,000,000). Candidate RNA polymers can be tested for suitability using any method, including, but not limited to, the assays described below.

However, the methods of the present invention are not limited to a particular RNA polymer. Any RNA polymer that inhibits the activity of RNase enzymes may be utilized. Suitable RNA polymers can be identified using one of several assays. For example, the ability of a given RNA polymer to inhibit RNase enzymes can be screened by adding varying amounts of the candidate polymer to a solution containing a RNA molecule of interest known to be capable of being degraded by RNase enzymes (e.g., MRNA) and the RNase enzyme. Suitable RNA polymers are those that prevent the RNA molecule of interest from being degraded, or reduce the level of degradation as determined by any suitable method, including, but not limited to, those described below.

For example, the samples can be electrophoresed and stained with an intercalating nucleic acid dye such as ethidium bromide. The level of protection provided by the candidate RNA polymer can be compared to reactions containing RNA polymers at a concentration (e.g., polyI:polyC; polyG) shown herein to inhibit RNase enzymes and to reactions containing no RNA polymers. Suitable RNA polymers are those that protect RNA molecules of interest from degradation. In some embodiments, suitable RNA polymers inhibit at least 25% of degradation relative to the level of degradation in the absence of a RNA polymer. In some embodiments, suitable RNA polymers inhibit at least 50% of degradation relative to the level of degradation in the absence of a RNA polymer. In some preferred embodiments, suitable RNA polymers inhibit at least 75% of degradation relative to the level of degradation in the absence of a RNA polymer. In some particularly preferred embodiments, suitable RNA polymers inhibit at least 90% of degradation relative to the level of degradation in the absence of a RNA polymer.

In addition, the level of degradation of an RNA of interest can be measured using the assay described in Example 12. In this example, the kinetics of hydrolysis of polyC by RNase enzymes is measured by an increase in absorbance at 255 nm. The rate of hydrolysis of the polyC RNA of interest in the presence or absence of the candidate RNA polymer is compared to controls containing no RNA polymer and a RNA polymer known to be inhibitory (e.g., polyG; See Example 12B-D). In some embodiments, suitable RNA polymers inhibit at least 25% of the hydrolysis of polyC relative to the level of hydrolysis in the absence of a RNA polymer. In some embodiments, suitable RNA polymers inhibit at least 50% of the hydrolysis of polyC relative to the level of hydrolysis in the absence of a RNA polymer. In some preferred embodiments, suitable RNA polymers inhibit at least 75% of the hydrolysis of polyC relative to the level of hydrolysis in the absence of a RNA polymer. In some particularly preferred embodiments, suitable RNA polymers inhibit at least 90% of the hydrolysis of polyC relative to the level of hydrolysis in the absence of a RNA polymer.

Another suitable assay for determining the ability of a candidate polymer to remove RNA binding enzymes from solution is described in Example 11. In this assay, the candidate RNA polymer is affixed to a solid support (e.g., a cyanogen bromide activated resin), hereinafter polymer:solid support. A solution known to contain an RNase enzyme or RNA binding protein is contacted with the polymer:solid support (e.g., by mixing the polymer:solid support with the solution or by passing the solution over the polymer:solid support packed in a column). The level of contaminating protein (e.g., RNase activity) in the solution is measured (e.g., using the method described in Example 12 or one of the additional methods described herein) prior to and after treatment with the polymer:solid support. In this example, the RNase activity after treatment with a candidate polymer:solid support is compared with the activity after treatment with a polymer:solid support known to remove RNases from solution (e.g., PolyG resin). In some embodiments, suitable RNA polymers inhibit at least 25% of the RNase activity of a solution relative to the level of activity prior to treatment with a RNA polymer resin. In some embodiments, suitable RNA polymers inhibit at least 50% of the RNase activity of a solution relative to the level of activity prior to treatment with a RNA polymer resin. In some preferred embodiments, suitable RNA polymers inhibit at least 75% of the RNase activity of a solution relative to the level of activity prior to treatment with a RNA polymer resin. In some particularly preferred embodiments, suitable RNA polymers inhibit at least 90% of the RNase activity of a solution relative to the level of activity prior to treatment with a RNA polymer resin.

A further assay for determining the ability of a candidate polymer to inhibit RNase enzyme activity is described in Examples 8-12. In these assays, RNase activity is measured by the change in absorption at 665 nm of a solution containing PolyA and methylene blue. The PolyA/methylene blue solution is incubated with an RNase enzyme and the absorbance is measured at various time points. The change in absorbance in the presence and absence of the candidate polymer is compared to the change in the presence of a polymer known to inhibit RNase activity (e.g., PolyG). In some embodiments, suitable RNA polymers inhibit at least 25% of the RNase activity of a solution relative to the level of activity in the absence of a RNA polymer. In some embodiments, suitable RNA polymers inhibit at least 50% of the RNase activity of a solution relative to the level of activity in the absence of a RNA polymer. In some preferred embodiments, suitable RNA polymers inhibit at least 75% of the RNase activity of a solution relative to the level of activity in the absence of a RNA polymer. In some particularly preferred embodiments, suitable RNA polymers inhibit at least 90% of the RNase activity of a solution relative to the level of activity in the absence of a RNA polymer.

B. Methods of Inhibiting RNase Enzymes

In some embodiments, the present invention provides methods of inhibiting RNAse enzymes. In some embodiments, the methods of the present invention are utilized to inhibit RNase enzymes, or remove, or decrease the amount of, an unwanted RNA binding protein from in vitro assays utilizing an RNA substrate (e.g., reverse-transcription reactions) or reactions that generate RNA molecules of interest (e.g., in vitro transcription reactions). The methods of the present invention are not limited to the reactions described herein. Indeed, it is contemplated that the compositions and methods of the present invention find use in any reaction containing an RNA of interest that is susceptible to degradation by RNase enzymes.

The present invention is not limited to the inhibition of a particular RNAse enzyme. Indeed, it is contemplated that the compositions and methods of the present invention find use in inhibiting a variety of RNase enzymes, including, but not limited to, RNase A, RNase H, RNase ONE, RNase B, RNase T₁, RNase T₂, RNase S, RNase from chicken liver, and RNase from Aspergillus clavatus.

Any suitable RNA polymer can be used in the methods of inhibiting RNase activity described herein. Suitable polymers include, but are not limited to those described in the sections above (e.g., including, but not limited to, polyA:polyU; polyC:polyG; polyC:polyI; polyI; polyC; polyG; polyA; poly(GU); poly(CU); poly(GI) and poly(CI), whole cell RNA such as HeLa RNA). Additional RNA polymers can be identified using any suitable screening assay, including, but not limited to, those described in the section above and the illustrative examples below. A suitable level of polymer to obtain the desired level of inhibition can also be determined using one of the screening assays described above and by the examples provided herein.

In some embodiments, RNase enzymes are inhibited by adding a suitable RNA polymer directly to the solution suspected of containing an RNase enzyme. For example, an enzymatic reaction utilizing an RNA substrate is treated by adding a suitable level of an RNA polymer. The polymer is ideally added to the reaction mixture prior to the addition of the component of the solution suspected of containing an RNase enzyme. Illustrative Examples 9-12 describe methods of inhibiting RNase enzymes in a solution containing an RNA molecule. Example 9 illustrates that the addition of polyI or polyG to a solution containing polyA inhibits degradation of the polyA by RNaseA. The polyI and polyG are more efficient at inhibiting RNaseA when they are pre-incubated with RNaseA (Example 10).

Alternatively, in preferred embodiments, the solution suspected of containing an RNase enzyme is incubated with a suitable polymer prior to the addition of remaining reaction components (e.g., components containing the RNA molecule to be protected). The methods of the present invention may also be utilized in a reaction where an RNA molecule is produced (e.g., an in vitro transcription reaction). In this case, the RNA polymers are initially added to the solution before the generation of the transcript of interest to prevent degradation of the RNA strand during and after generation.

Illustrative Example 12 demonstrates that polyG inhibits the kinetics of the degradation of a polyC substrate by both RNaseA and RNaseONE. Inhibition constants in the range of 10 nm-87 nM were obtained for the inhibition of RNaseA by polyG (Example 11B and 11C). Illustrative Example 11C indicates that the presence of spermidine in inhibition reactions increases the inhibition constant of polyG to 40 nM from 10 nM. Additionally, polyG was found to bind to RNaseONE at a stoichiometry of 3 RNaseONE molecules to one polyG molecule (See Example 13).

Previous work by others had indicated that chemical modification of RNA polymers is required for their use as inhibitors of RNA binding enzymes such as RNases (See e.g., U.S. Pat. No. 5,496,546). However, illustrative Example 12D demonstrated that substitution of FNDP-polyG for unmodified polyG did not improve the inhibition of RNaseOne. Thus, the present invention provides the advantage of using readily available unmodified RNA polymers, rather than the complex and expensive modified polymers.

In other embodiments, the inhibitory RNA polymers are attached to a solid support (e.g., a resin). The solid support is added directly as a slurry to a solution suspected of containing an RNA binding protein (e.g., RNase enzyme). The slurry, containing bound, contaminating RNase, is removed from the solution by centrifugation or allowed to settle out of solution. The supernatant (from which at least some RNA binding proteins have been removed due to binding to the resin) is then removed and used (e.g., in an enzymatic reaction). Alternatively, the solid support is packed into a column and the solution suspected of containing an RNase is passed over the column, retaining the RNAse enzyme and allowing the remainder of the solution to flow through the column. The solution (from which at least some RNA binding proteins have been removed due to binding to the resin) is then used in an enzymatic reaction.

In one illustrative example (Example 11), the treatment of RNase-containing solutions with RNA polymers attached to a resin was found to remove RNases from the solution. A column of polyG or polyI and polyG resin was found to remove RNaseB from solution. Likewise, a column of polyG, polyI, or polyI and polyG resin was found to remove RNaseA from solution. In addition, a column of polyG resin was found to remove RNaseONE Ribonuclease from solution.

II. Methods of Enhancing Enzymatic Reactions

In some embodiments, the present invention provides methods of enhancing enzymatic reactions (e.g., single tube RT-PCR). The present invention is not limited to any particular mechanism. Indeed, an understanding of the mechanism is not required to practice the invention. Nonetheless, it is contemplated that RNA polymers enhance RT-PCR reactions by removing the inhibitory effect of reverse transcriptase enzymes on PCR reactions. It is contemplated that after heat inactivation of the reverse transcriptase, it is bound by the RNA polymer. It is further contemplated that RNA polymers may additionally enhance enzymatic reactions (e.g., single tube RT-PCR) by inhibiting RNase enzymes that may degrade the mRNA templates used in such reactions.

In some embodiments of the present invention, dsRNA polymers (e.g., polyI:polyC; polyC:polyG, or polyA:polyU) are utilized to enhance enzymatic reactions. In other embodiments, ssRNA polymers (e.g., polyG; polyI, or polyC, polyA, poly(CU) are utilized to enhance enzymatic reactions. In some embodiments, two or more RNA polymers are combined. In some embodiments, additional RNA inhibitors (e.g., RNASIN Ribonuclease Inhibitor (Promega, Madison, Wis.)) are combined with a RNA polymer. The present invention is not limited to any particular mechanism. Indeed, an understanding of the mechanism is not required to practice the invention. Nonetheless, it is contemplated that ssRNA polymers such as polyI and polyC and polyG function in the enhancement of enzymatic reactions because they contain double stranded regions.

The present invention is not limited to any particular RNA polymer. Indeed, any suitable RNA polymer may be utilized, including, but not limited to, those described above. Additional suitable RNA polymers may be determined using any suitable assay, including, but not limited to, the assays described in Examples 1-7. Illustrative examples 1-7 describe the ability of RNA polymers to enhance RT-PCR reactions.

Illustrative Example 2 describes the effect of adding polyI:polyC to either a two-step (uncoupled; reverse transcription and PCR reactions performed separately) or one-step (coupled; all the components of both the reverse transcription and the PCR reaction are added initially) RT-PCR reaction. The addition of polyI:polyC increased the sensitivity (as measured in the lowest concentration of cells in which a signal could be detected) for both a one-step and two-step PCR reaction. The enhancement effect was found not to be due to enhancement of the PCR reaction (See Example 3).

Illustrative Example 4 describes the effect of a variety of RNA polymers on two-step RT-PCR. Poly(CI) and poly(CU) were found to slightly enhance the sensitivity of RT-PCR reactions. PolyI:polyC, polyG, polyI, poly(GU), and polyC:polyG were found to greatly increase the sensitivity of RT-PCR reactions. The concentration of polyI:polyC, polyG, or polyI used (within the range of 5 ng to 1 μg per 20 microliter volume) did not effect the level of enhancement (Example 5).

Illustrative Example 6 demonstrates that the RNA polymers of the present invention function to enhance RT-PCR in the presence of either avian myeloblastosis virus reverse transcriptase (AMV) RT or moloney murine leukemia virus (MMLV) RT.

Illustrative Example 7 describes the effect of polyI and polyG on one-step and two-step RT-PCR performed on whole cells. The enhancement of RT-PCR by these RNA polymers was compared to the enhancement of the RT-PCR reaction in the presence of RNASIN ribonuclease inhibitor. PolyI was shown to allow detection of an RT-PCR product when starting with as little as one cell's worth of template, which is comparable to the detection of an RT-PCR product in the presence of RNASIN ribonuclease inhibitor. PolyG also allowed successful RT-PCR from the whole cell lysates, but was not as sensitive as samples treated with RNASIN ribonuclease inhibitor or polyI under similar reaction conditions. PolyI also allowed successful amplification of the mRNA from whole cell lysates when the RT reaction was performed at an elevated temperature.

The methods of the present invention are not limited to the enhancement of RT-PCR reactions. Any reaction where it is advantageous to sequester or inhibit an RNA binding protein or an RNase enzyme may be enhanced by the methods of the present invention, including, but not limited to, RNA sequencing reactions, in vitro transcription reactions, in vitro translation reactions, RNA ligation reactions, use of nucleic acid arrays using RNA molecules, as well as drug screening, genomic, and diagnostic analysis techniques involving RNA.

III. Methods of Inhibiting Angiogenin

In some embodiments, the present invention provides pharmaceutical compositions comprising RNA polymers for the inhibition of RNase enzymes involved in cellular mechanisms (e.g., tumor growth). In some embodiments, the RNA polymers are targeted to tumors expressing angiogenin. Inhibition of angiogenin has been shown to reduce tumor growth (See e.g., Olson et al., PNAS, 92:442 [1995]; Gho and Chae, J. Biol. Chem., 272:24299 [1997]). The methods of the present invention are not limited to any one RNA polymer. Indeed, a variety of RNA polymers are contemplated. In some embodiments of the present invention, dsRNA polymers (e.g., polyI:polyC; polyC:polyG, or polyA:polyU) are utilized to inhibit the actions of angiogenin. In other embodiments, ssRNA polymers (e.g., polyG; polyI, polyA, poly(CU) or polyC) are utilized to inhibit the actions of angiogenin. Additionally, in some embodiments, two or more RNA polymers are combined. Additional suitable polymers may be identified using one of the screening assays described above or in the illustrative examples below. In some embodiments, the method described in illustrative Example 14 is used to identify suitable RNA polymers. Example 14 demonstrated that both polyG and RNASIN Ribonuclease Inhibitor inhibit the RNase activity of angiogenin in vitro. Methods for producing suitable pharmaceutical compositions are described below.

A. Pharmaceutical Compositions

The present invention provides pharmaceutical compositions that may comprise RNA polymers, pharmaceutically acceptable salts of RNA polymers, alone, or in combination with at least one other agent, such as a stabilizing compound, and may be administered in any sterile, biocompatible pharmaceutical carrier, including, but not limited to, saline, buffered saline, dextrose, and water.

The term “pharmaceutically acceptable salts” refers to physiologically and pharmaceutically acceptable salts of the compounds of the invention (i.e., salts that retain the desired biological activity of the parent compound and do not impart undesired toxicological effects thereto). Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine. The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations include, but are not limited to, alkaline, alkaline earth, ammonium and quaternary ammonium cations. Carbonates or hydrogen carbonates are also contemplated.

For RNA polymers, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The present invention also includes pharmaceutical compositions and formulations that include the RNA polymers of the present invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable. Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of nucleic acids (e.g., RNA polymers of the present invention) at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of RNA polymers.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more RNA polymers and (b) one or more other chemotherapeutic agents. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Two or more combined compounds may be used together or sequentially.

Dosing is dependent on severity and responsiveness of the disease state to be treated, with the course of treatment lasting from several days to several months, or until a cure is effected or a diminution of the disease state is achieved (e.g., shrinkage of tumor). For any compound used in the method of the invention, the therapeutically effective dose can be estimated initially from cell culture assays. Then, preferably, dosage can be formulated in animal models (particularly murine models) to achieve a desirable circulating concentration range that adjusts RNA polymer levels. Optimal dosing schedules can then be calculated from measurements of drug accumulation in the body of the patient. The administering physician can easily determine optimum dosages, dosing methodologies and repetition rates. Optimum dosages may vary depending on the relative potency of individual RNA polymers, and can generally be estimated. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compositions comprising RNA polymers are administered in maintenance doses, ranging from 0.01 μg to 100 g per kg of body weight, once or more daily, to once every 20 years.

Compositions comprising a compound of the invention formulated in a pharmaceutical acceptable carrier may be prepared, placed in an appropriate container, and labeled for treatment of an indicated condition. For RNA polymers, conditions indicated on the label may include treatment of the conditions discussed above (e.g., a tumor or other cancer).

B. Inhibition of Tumor Growth

In some embodiments of the present invention, a pharmaceutical composition comprising a suitable RNA polymer is administered to a mammal (e.g., a mouse, a rat, or a human) known to have a tumor or cancer. Any suitable RNA polymer may be utilized, including, but not limited to, those disclosed herein. Suitable dosages are determined as described above.

In preferred embodiments, administration of the pharmaceutical composition results in the shrinkage of the tumor or decreases the number of cancerous cells. In other preferred embodiments, tumor growth is halted upon administration of the RNA polymer. If the tumor or other cancer is not reduced, additional RNA polymers or increased dosages may be utilized in order to optimize the clinical effect. In some embodiments, RNA polymers are combined with other cancer therapies (e.g., chemotherapies, including, but not limited to those described above).

C. Drug Testing Applications

In some embodiments, the RNA polymers of the present invention find use in drug testing applications. For example, the compositions and methods of the present invention find use in the inhibition of RNase enzymes in cell culture (e.g., cells used for drug testing). Such methods are of particular use when the drug being testing is a nucleic acid (e.g., antisense RNA oligos). The therapeutic agent is protected from degradation so that it may be taken up by the cells and the therapeutic effect measured.

In other embodiments, the compositions and methods of the present invention are used to screen for novel RNA binding proteins involved in disease states. For example, a cell line (e.g., a tumor cell line) is contacted with a RNA polymer of the present invention. The RNA polymer is contacted with the cell in a pharmaceutical composition that permits entry into cells. The cells are lysed and the RNA polymers (with attached binding proteins) are recovered. The bound proteins in the tumor cell line are compared with the bound proteins in a control cell line, thus identifying novel RNA binding proteins involved in a disease state.

The present invention contemplates many other means of screening compounds. The examples provided above are presented merely to illustrate certain techniques available. One of ordinary skill in the art will appreciate that many other screening methods can be used.

IV. Kits

In some embodiments, the present invention provides kits comprising one or more of the components necessary for the inhibition of RNA binding proteins. In some embodiments, the kits further comprise a delivery system. In preferred embodiments, the kits comprise one or more RNA polymers. In some embodiments, the kits further comprise additional RNase inhibitors (e.g., RNASIN ribonuclease inhibitor).

In some embodiments, the kits of the present invention comprise one or more RNA polymers and a delivery system. In some embodiments, the delivery system is a solution (e.g., buffer) for delivery of the RNA polymer. In other embodiments, the delivery system is a solid support (e.g., a resin) and the RNA polymer is attached to the solid support. In yet other embodiments, the delivery system is a plastic reaction vessel (e.g., a tube or a microtiter plate) and the RNA polymer is attached to the plastic reaction vessel. The present invention is not limited to a particular delivery system. Indeed, any suitable delivery system may utilized. Such delivery systems will vary depending on the specific application and needs of the user.

In some preferred embodiments, the kits of the present invention further comprise controls. For example, an RNA substrate known to be digested by RNase enzymes. The control sample is incubated with the sample before and after treatment with the kit to ensure that unwanted RNases have been removed to a suitable level (See e.g., the description of suitable RNA polymers provided above).

In some embodiments, the kits further comprise additional components. For example, in some embodiments, the kits comprise additional RNase inhibitors, such as inhibitors made of protein (e.g., RNASIN ribonuclease inhibitor, Promega). In some embodiments, the kits comprise buffers or salts in order to provide optimal conditions for inhibition of RNases. In some embodiments (e.g., embodiments comprising RNA polymers affixed to solid supports), the kits further comprise buffers for washing and regenerating the solid support matrix (e.g., resin). In additional preferred embodiments, the kits further comprise instructions for utilizing the components of the kits (e.g., for using the kit to inhibit unwanted RNase enzymes).

Additionally, in some embodiments, the present invention provides methods of delivering kits or reagents for use in the inhibition of RNA binding enzymes (e.g., RNases) to customers. The methods of the present invention are not limited to a particular group of customers. Indeed, the methods of the present invention find use in the providing of kits or reagents to customers in many sectors of the biological and medical community, including, but not limited to customers in academic research labs, customers in the biotechnology industry, and customers in governmental labs. The methods of the present invention provide for all aspects of providing the kits or reagents to the customers, including, but not limited to, marketing, sales, delivery, and technical support.

In some embodiments of the present invention, quality control (QC) and/or quality assurance (QA) experiments are conducted prior to delivery of the kits or reagents (e.g., RNA polymers of the present invention) to customers. Such QC and QA techniques typically involve testing the polymers in experiments similar to the intended commercial uses (e.g., using assays similar to those described herein to test the inhibitory activity of the polymers). Testing may include experiments to determine shelf-life of products and their ability to withstand a wide range of solution conditions (e.g., temperature, pH, light, etc.).

In some embodiments of the present invention, the inhibitors are demonstrated to customers prior to sale (e.g., through printed or web-based advertising, demonstrations, etc.) indicating the use of the polymers of the present invention in inhibiting RNases or RNA binding proteins. However, in some embodiments, customers are not informed of the presence or use of the polymer in the product being sold. In such embodiments, sales are developed through the improved and/or desired function of the product (e.g., kit) rather than through knowledge of why or how it works. For example, the polymers may be sold covertly in a reaction buffer of vessel that finds use in a method where the polymers of the present invention are useful (e.g., RT-PCR).

The kits of the present invention thus provide improved methods of inhibiting unwanted RNA binding by RNA-binding proteins or inhibiting RNase enzymes. Such improved uses provide incentive to customers to purchase the kits. Accordingly, in some embodiments, sales and marketing efforts present information about the improved properties. In other embodiments, such mechanistic information is withheld from marketing materials. In some embodiments, customers are surveyed to obtain information about the type of buffer and delivery system that most suits their needs. Such information is useful in both the design of the components of the kit and the design of marketing efforts.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: M (Molar); μM (micromolar); mM (millimolar); nM (nanomolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micron); nm (nanometers);° C. (degrees Centigrade); U (units); min. (minutes); sec. (seconds); % (percent); kb (kilobase); bp (base pair); PCR (polymerase chain reaction); RT (reverse transcriptase); RT-PCR (reverse transcriptase-PCR); BSA (bovine serum albumin); Sigma (Sigma Chemical Co., St. Louis, Mo.); Boehringer Mannheim (Boehringer Mannheim, Corp., Indianapolis, Ind.); Pharmacia (Pharmacia, Peapack, N.J.); R&D Systems (R&D Systems, Minneapolis, Minn.); and Promega (Promega Corp., Madison, Wis.).

### EXAMPLE 1

### Effect of Hela Total RNA on Template Sensitivity

This Example describes the effect of HeLa cell RNA on template-detection sensitivity in two-step RT-PCR performed on a poly(A)+ transcript.

A. Reverse Transcription Reactions

Kanamycin poly(A)+ MRNA (Promega, C138A) target was titrated by 1:100 dilutions in nuclease-free water so that each 20 μl reaction contained from 10¹² to 10² copies of poly(A)+ transcript. Blank control reactions contained only nuclease-free water. Each RNA target dilution was combined with oligo d(T)₁₅ primer (Promega, C110A) so that 3 μl contained the designated amount of copies of RNA target and 0.5 μg of primer. These combinations were scaled-up into 10× to 25× batches so that aliquots of one target:primer combination could be sampled into each reaction to minimize sampling variability. The target:primer combinations were heated at 70° C. for 10 min, and then quick chilled at 4° C. The reverse transcription (RT) reaction mixes were set up so that individual 20 μl reactions contained the components listed in Table 1. Seventeen microliter aliquots of each RT mix were dispensed to reaction tubes at 4° C. Reaction tubes received 3 μl aliquots of each batch of denatured RNA target:primer combination on ice. Reverse transcription reactions were performed as follows: 25° C. for 5 min (annealing), 42° C. for 60 min (cDNA synthesis), and then heat inactivated at either 95° C. for 5 min (a) or 70° C. for 15 min (b,c,d), and finally chilled at 4° C. for one hour (a,b), −20° C. for 1 hour (c), or -70° C for 1 hour (d).

B. PCR Reactions

An amplification mix was produced to provide 80 μl additions to each 20 μl cDNA reverse transcription product for a total PCR reaction volume of 100 μl. The final concentration of components in the PCR reactions was: 1× PCR buffer (Promega, M190A); 2 mM MgCl₂; 0.2 mM each dNTP; 0.15 μM upstream primer (Promega, A109A; 5′ GCCATTCTCACCGGATTCAGTCGTC 3′; SEQ ID NO:1); 0.15 μM downstream control primer (Promega, A110A; 5′ AGCCGCCGTCCCGTCAAGTCAT 3′; SEQ ID NO:2); 2.5 U Taq DNA polymerase; and 2 drops mineral oil. The amplification mix was prepared on ice and dispensed into the 20 μl cDNA reaction on ice. PCR was performed as follows: 94° C. for 2 min; (94° C. for 1 min, 52° C. for 1 min, 72° C. for 2 min)×35; extension at 72° C. for 5 min; and storage at 4° C. overnight. Samples (10μl of each PCR reaction) were run into an agarose gel and stained with intercalating fluorescence dye. The gel was visualized using a MD Fluorimager.

Table 2 shows the levels of detection sensitivity obtained under the various conditions tested, i.e. the number of RNA molecules of poly(A)+ mRNA present in the sample that were detectable by the method of the assay. Sensitivity limits of 10¹⁰ copies of RNA per reaction were observed for all heat inactivation and cold storage methods tested. A dramatic improvement in sensitivity to 10² was seen when reactions included 100 ng of HeLa total RNA (Reactions III and IV).

TABLE 1
Example 1 Reaction Conditions

| | |
|---|---|
| Rxn I | 200 U MMLV RT RNase H(−) point mutant (Promega, |
| | M368A); 50 mM Tris (pH 8.3); 75 mM KCl, 3 mM MgCl₂; |
| | 10 mM DTT; 0.5 mM each dNTP |
| Rxn II | 200 U MMLV RT RNase H(−) point mutant; 33 mM Tris |
| | (pH 8.3); 50 mM KCl; 6 mM MgCl₂; 10 mM DTT; 10% |
| | sucrose; 0.5 mM each dNTP |
| Rxn III | 200 U MMLV RT RNase H(−) point mutant; 33 mM Tris |
| | (pH 8.3); 50 mM KCl; 6 mM MgCl₂; 10 mM DTT; 10% |
| | sucrose; 0.5 mM each dNTP; 100 ng Hela Total RNA |
| Rxn IV | 50 U RT formulation (Promega, M380A); 33 mM Tris |
| | (pH 8.3); 50 mM KCl; 6 mM MgCl₂; 10 mM DTT; 10% |
| | sucrose; 0.5 mM each dNTP; 100 ng Hela Total RNA |

TABLE 2
RNA Target Sensitivity of Example 1

| Rxn cmpnts./ | | | | |
|---|---|---|---|---|
| RT inactivation | | | | |
| and chilling | | | | |
| conditions | (a.) | (b.) | (c.) | (d.) |
| I | 10¹⁰ | 10¹⁰ | 10¹⁰ | 10¹⁰ |
| II | 10⁸ | 10⁸ | 10⁶ | 10⁶ |
| III | 10² | 10² | 10² | 10² |
| IV | 10² | 10² | 10² | 10² |

### EXAMPLE 2

### Effect of polyI:polyC and Sucrose on Target Sensitivity

This Example describes the effect of polyI:polyC RNA on substrate sensitivity in both single-tube and two-tube PCR performed on a poly(A)+ transcript.

A. Reverse Transcription Reactions

Kanamycin RNA transcript target was titrated by 1:100 dilutions in nuclease-free water so that each 20 μl reaction contained from 10¹² to 10² copies of poly(A)+ transcript. Blank control reactions contained only nuclease-free water. Samples of each RNA target dilution representing from 10⁴ to 10² copies were combined with oligo d(T)₁₅ primer so that 3 μl contained the designated number of copies of kanamycin RNA transcript and 0.5 μg of primer. These combinations were scaled-up into 35× batches so that aliquots of one target:primer combination could be sampled into each reaction to minimize sampling variability.

The target:primer combinations were denatured at 70° C. for 10 min, then quick chilled at 4° C. RT reaction mixes were set up so that individual 20 μl reactions contained the components listed in Table 3. Then 17 μl aliquots of each RT mix, containing everything except the target/primer mix, were dispensed in duplicate to each reaction tube on ice. Reaction tubes received 3 μl aliquots of each batch of denatured RNA target:primer combination (10⁴; 10^{3;} and 10² copies of RNA target) at 4° C.

Each 20 μl reverse transcription reaction was incubated at 25° C. for 5 min (annealing), followed by 42° C., 46° C., or 50° C. for 60 min (cDNA synthesis), and then heat inactivated for 15 min at 70° C. and stored overnight at −70° C. (Rxns I-V) or immediately used in a PCR reaction (VI).

B. PCR Reactions

Amplification mix was produced to provide sufficiently for 80 μl additions to each 20 μl cDNA reverse transcription product for a total PCR reaction volume of 100 μl. The final concentration of components in the PCR reactions were: 1× PCR buffer (Promega M190); 2 mM MgCl₂; 0.2 mM each dNTP; 0.15 μM upstream control primer (Promega A109A); 0.15 μM downstream control primer (Promega, A110A); 2.5 U Taq DNA polymerase (Promega, M166A); and 2 drops of mineral oil. The amplification mix was prepared on ice and dispensed into the 20 μl cDNA reaction on ice (Rxns. I-V). PCR was performed as follows: 95° C. for 2 min; (95° C. for 1 min, 52° C. for 1 min, and 72° C. for 2 min)×35; extension of 72° C. for 5 min; and storage at 4° C. overnight.

Samples (10 μl of each PCR reaction) were run into an agaorse gel in 1× and stained with intercalating fluorescence dye. The gel was visualized using a MD Fluorimager.

Table 4 shows the levels of sensitivity obtained under the various conditions. Weak reactions were observed in reactions I-III, with 10⁴ copies of RNA target barely detectable. The addition of polyI:polyC high molecular weight dsRNA increased the sensitivity of the reaction greatly. A strong reaction at 10² copies of target RNA was observed in the presence of polyI:polyC (Reactions IV-V). The one-step reaction (reaction VI), in which all of the PCR components are initially present in the reverse-transcriptase reaction, showed a moderate accumulation of product at 10² copies of target and strong reactions at higher levels of target RNA.

TABLE 3
Example 2 Reaction Conditions

| | |
|---|---|
| Rxn I | 50 U MMLV RT RNase H(−) point mutant (Promega, |
| | M368A); 50 mM Tns (pH 8.3); 75 mM KCl, 3 mM MgCl₂; |
| | 10 mM DTT; 0.5 mM each dNTP |
| Rxn II | 50 U MMLV RT RNase H(−) point mutant; 33 mM |
| | Tris (pH 8.3); 50 mM KCl; 6 mM MgCl₂; 10 mM DTT; 10% |
| | sucrose; 0.5 mM each dNTP |
| Rxn III | 50 U MMLV RT RNase H(−) point mutant; 33 mM |
| | Tris (pH 8.3); 50 mM KCl; 6 mM MgCl₂; 10 mM DTT; 10% |
| | sucrose; 0.5 mM each dNTP |
| Rxn IV | 50 U MMLV RT RNase H(−) point mutant; 33 mM |
| | Tris (pH 8.3); 50 mM KCl; 6 mM MgCl₂; 10 mM DTT; |
| | 0.5 mM each dNTP; 100 ng polyI:polyC high molecular |
| | weight dsRNA (Sigma) |
| Rxn V | 50 U MMLV RT RNase H(−) point mutant; 33 mM |
| | Tris (pH 8.3); 50 mM KCl; 6 mM MgCl₂; 10 mM DTT; |
| | 0.5 mM each dNTP; 10% sucrose; 100 ng pI:pC high molecular |
| | weight dsRNA |
| Rxn VI | 50 U MMLV RT RNase (−) point mutant; 33 mM |
| | Tris (pH 8.3); 50 mM KCl; 2 mM MgCl₂; 10 mM DTT; |
| | 0.5 mM each dNTP; 10% sucrose; 100 ng pI:pC high molecular |
| | weight dsRNA; 2.5 U Taq DNA Polymerase; 0.75 μM of |
| | upstream primer and 0.75 μM of downstream primer |

TABLE 4
RNA Target Sensitivity In Example 2

| Rxn cmpnts./ | | | |
|---|---|---|---|
| Conditions | 42° C. | 46° C. | 50° C. |
| I | no signal | no signal | 10³ (Weak) |
| II | 10⁴ (weak) | 10⁴ (weak) | no signal |
| III | 10⁴ (weak) | 10⁴ (weak) | no signal |
| IV | 10² (very strong) | 10² (very strong) | 10² (very strong) |
| V | 10² (very strong) | 10² (very strong) | 10² (very strong) |
| VI | 10² (moderate) | not analyzed | not analyzed |

### EXAMPLE 3

### Effect of polyI:polyC on PCR

This example was designed to determine if the enhancement of RT-PCR by polyI:polyC is due to enhancement of the PCR step. DNA template was prepared with the Promega RT-PCR System (A1250) according to manufacturer's instructions. The template (approximately 10¹² copies) was serially diluted by a factor of 10. Final dilutions were as follows: 10⁻², 10⁻³, 10⁻⁴, 10⁻⁵, 10⁻⁶, 10⁻⁷, 10⁻⁸, 10⁻⁹, 10⁻¹⁰, and 10⁻¹¹. Each DNA concentration was combined, in 100 μl reaction, to contain a final of: 1). 1× PCR buffer (Promega M190); 2 mM MgCl₂, 0.2 mM each dNTP, 0.15 μM upstream and downstream primers (Promega, A109 and A110), 2.5 U Taq DNA polymerase (Promega, M166A); and 2). 1× PCR buffer; 2 mM MgCl₂, 0.2 mM each dNTP, 0.15 μM upstream and downstream primers as listed above, 2.5 U Taq DNA polymerase, 100 ng polyI:polyC. Reaction mix (99 μl) was combined with 1 μl of each of the DNA dilutions on ice.

PCR reactions were performed as follows: 94° C., 2 min; (94° C., 1 min; 52° C., 1 min; 75° C., 2 min)×35 followed by extension of 72° C. for 5 min; and 4° C. overnight. Samples (5 μl of each PCR reaction) were analyzed on an agarose gel with intercalating fluorescence dye. The gel was visualized using a MD Fluorimager.

Results indicated that the presence or absence of polyI:polyC had no detectable effect on the intensity, band quality or sensitivity of the PCR reaction when reverse transcriptase was not present. Product was observed at all dilutions of template, with no increased sensitivity in the presence of polyI:polyC.

### EXAMPLE 4

### Effect of Different RNA Polymers on RT-PCR

This example describes the effect of a variety of RNA polymers on the enhancement of RT-PCR reactions using three different MMLV reverse transcriptase formulations. One microgram quantities of seventeen different RNA polymers were tested individually in RT-PCR reactions to note which facilitated the production of RT-PCR amplification product.

The RT reaction mix batches were assembled so that each 20 μl reaction would consistently contain 100 copies of full length, 1.2 Kb poly(A)+ MRNA target and 0.5 μg of oligo d(T) primer which had been heat denatured and chilled. The final RT reaction conditions were 33 mM Tris-HCl (pH 8.3), 50 mM KCl, 6 mM MgCl2, 10 mM DTT, 0.5 mM each dNTP. The original batch of reaction mix was divided into ten reaction subsets. To each individual subset, one of the seventeen different RNA polymers was added, 10 μg of RNA polymer added to each. Each individual RNA polymer reaction mix batch was subsequently divided into three reaction volumes. For each set of three, each subset received a different reverse transcriptase. The three-reaction RT mixes were aliquoted into individual RT reactions so that the RNA polymer effect was tested with 200 U MMLV RNase H(−) point mutant (Promega M368A); 200 U MMLV RNase H(+) (Promega M170A); and 50 U MMLV RNase H(−) point mutant (Promega M380). Reverse transcription reactions were performed as follows: annealing for 5 minutes at 25° C., cDNA synthesis for 60 minutes at 42° C., inactivation at 70° C. for 15 minutes and storage overnight at −70° C.

Eighty microliters of a PCR mix was added to each 20 μl RT reaction. Final concentrations were: 1× PCR buffer (Promega M190A), 2 mM MgCl₂, 0.2 mM each dNTP, 0.15 μM each primer (Promega A109A and A110A), 2.5 U Taq DNA polymerase, and 2 drops of mineral oil. PCR reactions were performed as follows: 94° C., 2 min (94° C., 1 min; 60° C., 1 min; 68° C., 2 min)×35 cycles; extension of 72° C. for 5 min; and storage at 4° C. overnight. Samples (5 μl of each PCR reaction) were run into an agarose gel and stained with intercalating fluorescence dye. The gel was visualized using a MD Fluorimager.

No detectable RT-PCR products were observed in the absence of RNA polymer or in the presence of the following RNA polymers (for all RT enzymes tested): polyA (Amersham), polyC (Amersham and Sigma), poly(IU) (Sigma). Faint RT-PCR products were observed in the presence of the following RNA polymers: poly(CI) (Sigma) with all RT enzymes; poly(CU) (Sigma) with Promega's M170 RT enzyme. Robust RT-PCR products were detected in the presence of the following RNA polymers: HeLa Total RNA with Promega's M368 RT enzyme; tRNA (Sigma) with Promega's M368 RT enzyme; 5S+16S+23S combination RNA (Boehringer Mannheim) with Promega's M368 RT enzyme; polyI:polyC (Amersham and Sigma) with all RT enzymes tested; polyG (Sigma) with all RT enzymes tested; polyI (Sigma) with all RT enzymes tested; poly(GU) (Sigma) with all RT enzymes tested; polyC:polyG (Sigma) with all RT enzymes tested. These results indicate that a wide variety of RNA polymers can be used in the enhancement of RT-PCR reactions.

### EXAMPLE 5

### The Effect of RNA Polymer Concentration on RT-PCR

The purpose of this experiment was to determine how addition of varied concentration of several RNA polymers effect RT-PCR reactions. A limiting amount of starting polyA(+) target was used as template for the reverse transcription reaction. It was previously demonstrated that RT-PCR using MMLV H+ reverse transcription of 100 copies of target RNA in the absence of RNA polymer produces no detectable product. This concentration was thus used in the following experiment.

Target (1.2 Kb kanamycin RNA transcript) was diluted through 100-fold steps in nuclease-free water so that each 20 μl RT reaction contained a standardized amount of 100 copies of full length, polyA(+) transcript. Batch combinations of RNA target, oligo d(T) primer and water were assembled in thin walled reaction tubes. The RNA and primer combination tube was incubated in a 70° C. controlled temperature block to thermally denature and then quickly transferred to ice.

RNA polymers used were polyI:polyC (potassium salt from Amersham), polyG (sodium salt from Sigma), and polyI (sodium salt from Sigma). Dilutions from 2.5 mg/ml stocks were prepared. 5 μl of each RNA polymer dilution was dispensed to the appropriate, autoclaved thin wall reaction tube on ice.

A 2× concentration of RT mix was assembled and 10 μl was dispensed into each of the tubes containing 5 μl RNA polymer. Tubes were kept on ice. The RNA target and primer were added last. Final concentrations were 33 mM Tris-HCl (pH 8.3), 50 mM KCl, 10 mM DTT, 6 mM MgCl₂, 0.5 mM each dNTP, 0.5 μg oligo-d(T) primer, 100 copies full length 1.2 Kb Kanamycin transcript RNA, 200 U MMLV reverse Transcriptase (Promega Corp., M170A), and either 1 ng, 5 ng, 25 ng, 50 ng, 75 ng, 100 ng, 200 ng, 300 ng, 400 ng, 500 ng, 600 ng, 800 ng or 1 μg RNA polymer.

Reverse transcription reactions were performed as follows: annealing for 5 min at 25° C., cDNA synthesis for 60 min at 37° C., inactivation. at 70° C. for 15 minutes, chill to 4° C., and storage overnight at −70° C.

PCR amplification mix was prepared so that 80 μl of mix could be added directly to the 20 μl RT reactions. The final amplification reaction conditions were: 1× PCR reaction buffer (Promega, M190), 2 mM MgCl₂, 0.2 mM each dNTP, 0.15 μM each upstream and downstream primers (A109 and A110), and 2.5 U Taq DNA polymerase (Promega, M166). Eighty microliters of the amplification reaction was added to 20 μl of the RT reaction product on ice. PCR reactions were performed as follows: 94° C. for 2 min; (94° C. for 1 min; 60° C. for 1 min; 68° C. for 2 min)× 35; extension of 72° C. for 5 min; and 4° C. overnight. Samples (5 μl of each PCR reaction) were run on an agarose gel and stained with intercalating fluorescence dye. The gel was visualized using a MD Fluorimager.

Each of the RNA polymers enhanced the detection of target RNA at all concentrations tested. These results indicate that a variety of RNA polymers and polymer concentrations can enhance enzymatic reactions such as RT-PCR.

No amplification products were observed when no RNA polymer was present in the RT-PCR reactions. Each of the RNA polymers enhanced the detection of target RNA within the concentration range tested, with faint amplification product noted at the concentration of 5 ng RNA polymer and very strong amplification product noted at 1 migrogram RNA polymer per reaction. These results indicate that a variety of RNA polymers and polymer concentrations can enhance enzymatic reactions such as RT-PCR.

### EXAMPLE 6

### The Effect of RNA Polymer in RT-PCR using AMV Reverse Transcriptase

The purpose of this experiment was to determine if the enhancing effect of RNA polymer in a RT-PCR reaction was unique to interaction with MMLV reverse transcriptase. The experiment was designed to test the effect of the addition of an RNA polymer to a RT-PCR that employed a reverse transcriptase other than MMLV reverse transcriptase. Single-tube RT-PCR was performed using AMV reverse transcriptase and Tfl DNA polymerase in reaction conditions optimized for this enzyme combination.

Kanamycin RNA transcript target was titrated by 1:100 dilutions in nuclease-free water so that each 50 μl reaction contained from 10¹⁰ to 10² copies of poly(A)+ transcript. Blank control reactions contained only nuclease-free water. Samples of each RNA target dilution representing 10¹⁰ to 10² or 0 copies were combined with upstream (SEQ ID NO: 1) and downstream (SEQ ID NO: 2) gene-specific primers so that 5 μl contained the designated number of copies of kanamycin RNA transcript and 50 pmoles of each primer. These combinations were scaled-up into 10× batches so that aliquots of one target:primer combination could be sampled into each reaction to minimize sampling variability. The target:primer combinations were heated at 70° C. for 10 minutes, then quick chilled in on ice.

RT-PCR reaction mixes minus target:primer were set up in volume excess so that the resulting individual 50 μl reactions contained the following components. Each RT-PCR reaction contained standardized 1× AMV/Tfl Reaction Buffer (from Promega ACCESS RT-PCR System, Catalog#A1250), 2mM MgSO₄, 0.2 mM of each dNTP, 50 units of AMV Reverse Transcriptase (Promega, Catalog #M5101), and 5 units Tfl DNA polymerase (component in Promega, Catalog #A1250). Two 13× batches of RT-PCR reaction mix were prepared on ice. To each mix, the RNA polymer polyI:polyC or water was added so that each 50 μl RT-PCR reaction contained the variable components defined in sets as follows:

Set I no polyI:polyC

Set II 2 μg/ml polyI:polyC

Each target:primer point was tested in duplicate. Aliquots of 45 μl of each of the RT-PCR reaction mixes were distributed to PCR tubes on ice. Duplicate reaction tubes received 5 μl aliquots of each denatured RNA target:primer combination so that each set tested the system's sensitivity in RT-PCR over the range of 10¹⁰ to 10² and 0 copies of RNA transcript.

Each 50 μl RT-PCR reaction was incubated in a thermocycler at 25° C. for 5 minutes (annealing), 48° C. for 60 minutes (cDNA Synthesis), 2 minutes at 95° C. (heat inactivation), 35 cycles of 95° C. for 1 minutes, 60° C. for 1 minute, 72° C. for 2 minutes (PCR Amplification) and 72° C. for 5 minutes (extension). The reaction tubes were then held at 4° C. until analysis.

Samples (10 μl of each RT-PCR reaction) were run on an agarose gel and stained with intercalating fluorescence dye. The gel was then visualized using a MD fluorimager. The gel analysis showed the level of sensitivity obtained under the various conditions. The addition of 2 μg/ml polyI:polyC high molecular weight double-stranded RNA dramatically increased the sensitivity of the reactions. A consistently strong reaction at 10² copies of target RNA was observed in the presence of 2 μg/ml polyI:polyC when 50 units of AMV reverse transcriptase and 5 units of Tfl DNA polymerase were present in the reaction. Without the polyI:polyC, the RT-PCR reactions did not yield any reaction product below the target level of 10⁶ copies per reaction. This experiment demonstrates that AMV reverse transcriptase responds to RT-PCR enhancement by addition of RNA polymer such as polyI:polyC in a manner similar to that observed using MMLV reverse transcriptase.

### EXAMPLE 7

### Inhibition of RNase Enzymes in RT PCR Reactions

This example evaluates the utility of polyG or polyI attached to resins in enhancing single-cell RT-PCR reactions performed on whole eukaryotic cell lysates. The effect of the resins was compared to RNASIN ribonuclease inhibitor (Promega). In addition, one step RT-PCR reactions (both RT and PCR reaction components in one reaction tube) were compared to two-step reactions (RT and PCR reactions performed separately). Tables 5 and 6 outline the number of cells and the RNase inhibitor added to each reaction.

A. One Step RT-PCR

RT-PCR reactions were conducted without prior RNA isolation. Exponentially growing K562 (human erythroleukemia cell line) cells were washed once in cold 1× PBS, then serially diluted in cold 1× PBS to 0, 0.2, 1.0, 2.0, 4.0, and 10.0 cells/μl. An aliquot of each cell dilution (5 μl) was then placed in duplicate 0.5 ml microfuge tubes and the following were added to each set of tubes: 5 μl 2× RNASIN ribonuclease inhibitor solution (2 μl RNASIN, 18 μl 0.15 M NaCl/10 mM Tris-HCl (pH 8.0)/5 mM DTT), 1 μl of either PolyG, PolyI, or PolyG+PolyI resin (see example 11) plus 4 μl 1× PBS, or 3 μl of either PolyG, PolyI, or PolyG+PolyI resin plus 2 μl 1× PBS. The tubes of cells plus RNASIN ribonuclease inhibitor or resin were then frozen at −70° C. and thawed at room temperature to lyse the K562 cells. The samples containing resin were conducted in quadruplicate, such that after the freeze/thaw step, one set of duplicate samples was spun at 14,000 rpm in a microfuge to pellet the resin. The supernatant was decanted and used in the RT-PCR reaction. The other set of reactions were not microfuged (the resin was carried over into the RT-PCR reaction). Experimental conditions are shown in Table 6. Each reaction condition was performed at each cell concentration given in Table 5.

Each sample (the entire 10 μl) was then added to RT-PCR reactions using the Access RT-PCR kit and a bcr/abl primer pair (forward: 5′ GGAGCTGCAGATGCTGACCAAC 3′; SEQ ID NO:3 and reverse: 5′ TCAGACCCTGAGGCTCAAAGTC 3′; SEQ ID NO:4). The final concentration of reaction components was: 1× RT-PCR buffer, 200 μM each dNTP, 2 mM MgSO₄, 50 pmoles forward and reverse bcr/abl primers, 5 units AMV RT, and 5 U Tfl DNA polymerase. The reactions were cycled using the following parameters: 45 min at 48° C.; 2 min at 95° C.; 40 cycles of 94° C. for 30 sec, 60° C. for one min, and 72° C. for 1 min; 1 cycle of 7 min at 72° C., and then stored at 4° C. Aliquots of each reaction (6 μl) were then analyzed on a 1.8% agarose/1× TAE gel and visualized with ethidium bromide staining.

Results are shown in Table 6. The addition of RNASIN ribonuclease inhibitor freeze medium to the cells during lysis allowed for the sensitive detection of the bcr/abl signal, down to as low as 1 cell, with increasing signal intensity with increasing cell number. The addition of 1 μl, but not 3 μl of the poly-G resin allowed detection of the bcr/abl signal down to approximately 1 to 10 cells (with or without spin). The signal in the presence of polyG was weaker than with RNASIN. Addition of either 1 μl or 3 μl of PolyI allowed detection to a level comparable to RNASIN, and provided an increased level of sensitivity when not removed prior to PCR reaction. The polyI and polyG combination of resin did not result in a substantial increase in sensitivity. The results indicate that RNA polymers can replace RNASIN in single-tube, whole cell RT-PCR.

B. Two Step RT-PCR

Two-step RT-PCR reactions were also performed on the previously described K562 cell samples. The cell sample dilutions were added to 6.75 μl nuclease-free water, 1 μl oligo-dT (0.5 μg), 5 μl 5× MMLV RT buffer, 1.25μl 10 mM dNTP mix, and 1 μl (50 U) MMLV H-RT point mutant in a final reaction volume of 25 μl. The reactions were incubated at 37° C. for 10 min, 55° C. for 50 min, and 70° C. for 15 min. An aliquot of each RT reaction (10 μl) was added to a subsequent PCR amplification reaction containing 1× thermophilic polymerase buffer (Promega Corp., M190A), 1.5 mM MgCl₂, 200 μM each dNTP, 50 pmoles forward (5′ TCATGAAGTGTGACGTTGACATCCGT 3′; SEQ ID NO:5) and reverse (5′ CCTAGAAGCATTTGCGGTGCACGATG 3′; SEQ ID NO:6) beta-actin primers, and 2 U of ampli-Taq DNA polymerase.

The reactions were cycled using the following parameters: 3 minutes at 95° C.; (94° C. for 30 sec, 60° C. for one min, and 70° C. for 1 min) x 40; 5 min at 68° C., and stored at 4° C. Aliquots of each reaction (6 μl) were then run on an agarose and visualized with ethidium bromide staining.

Results indicated that only the polyI resin resulted in a signal. Signal was obtained at a cell concentration of 10-50 cells. As the reaction was conducted at 55° C., polyI has an advantage over RNASIN ribonuclease inhibitor, which is denatured at temperatures over 50° C.

TABLE 5
Number of Cells Used in Example 7

| | Number of K562 Cells |
|---|---|
| Sample Number | (Cells/μl) |
| 1 | 0 |
| 2 | 1 |
| 3 | 5 |
| 4 | 10 |
| 5 | 20 |
| 6 | 50 |

TABLE 6
Reaction Conditions for Example 7

| | | | Spin After | Results |
|---|---|---|---|---|
| Sample Name | Additive | Amount | Lysis | (Sensitivity) |
| A | RNASIN | 5 μl | No | 1 Cell |
| | ribonuclease | | | |
| | inhibitor | | | |
| B | PolyG Resin | 1 μl | No | 1-10 |
| C | PolyG Resin | 3 μl | No | no amplification |
| | | | | product visible |
| D | PolyG Resin | 1 μl | Yes | 1-10 |
| E | PolyG Resin | 3 μl | Yes | no amplification |
| | | | | product visible |
| F | PolyI Resin | 1 μl | No | 1-10 |
| G | PolyI Resin | 3 μl | No | 1-10 |
| H | PolyI Resin | 1 μl | Yes | 1-10 |
| I | PolyI Resin | 3 μl | Yes | 1-10 |
| J | PolyG + PolyI | 1 μl | No | no amplification |
| | Resin | | | product visible |
| K | PolyG + PolyI | 3 μl | No | no amplification |
| | Resin | | | product visible |
| L | PolyG + PolyI | 1 μl | Yes | 10-20 |
| | Resin | | | |
| M | PolyG + PolyI | 3 μl | Yes | no amplification |
| | Resin | | | product visible |

### EXAMPLE 8

### RNase Digestion Monitored by Methylene Blue

This example describes the use of methylene blue to measure the digestion of polyA by RNase A. RNase A activity was measured by the absorption at 665 nm of a solution containing PolyA and methylene blue. A PolyA stock (20 μg/ml) containing 1 mg/100 ml methylene blue was prepared in 100 mM NaOAc at pH 6.0 and aliquoted into 5 tubes. A 1 mg/ml RNase A solution was prepared in 100 mM NaOAc, pH 6.0 and added to the polyA/methylene solution so that RNase A was present at 0, 5, 10, 15, or 20 μg/ml. Likewise, a polyI (Sigma Corp., Cat. P4404) stock (20 μg/ml) containing 1 mg/100 ml methylene blue was prepared in 100 mM NaOAc at pH 6.0, aliquoted into 5 tubes, and the same amounts of RNase A was added to these solutions. The reactions were incubated up to 60 minutes at room temperature and the absorbance was measured at 665 nm at 1, 15, 30, 45, and 60 minutes. Results are shown in Table 7 below.

TABLE 7
OD 665 In the Presence of Methylen Blue

| | Time | RNase A (μg/ml) | | | | |
|---|---|---|---|---|---|---|
| Polymer | (min) | 0 μg | 5 μg | 10 μg | 15 μg | 20 μg |

| | | | | | | |
|---|---|---|---|---|---|---|
| PolyA | 0 | 1.0221 | 1.0513 | 1.0626 | 1.0812 | 1.0854 |
| PolyA | 15 | 1.0568 | 1.0733 | 1.1193 | 1.2089 | 1.2939 |
| PolyA | 30 | 1.0803 | 1.1295 | 1.3105 | 1.4778 | 1.6227 |
| PolyA | 45 | 1.0698 | 1.1952 | 1.4651 | 1.6621 | 1.8066 |
| PolyA | 60 | 1.0813 | 1.2676 | 1.5799 | 1.8038 | 1.8998 |
| PolyI | 0 | 1.2352 | 1.2335 | 1.2352 | 1.231 | 1.2295 |
| PolyI | 15 | 1.2061 | 1.1898 | 1.1747 | 1.1803 | 1.1656 |
| PolyI | 30 | 1.2048 | 1.1796 | 1.1562 | 1.1615 | 1.1603 |
| PolyI | 45 | 1.1996 | 1.1555 | 1.1558 | 1.168 | 1.1407 |
| PolyI | 60 | 1.2076 | 1.1571 | 1.1562 | 1.171 | 1.1359 |

The experimental results demonstrate that polyA digestion by RNAse A enzyme can be followed using methylene blue dye at an absorbance of 665 mn. The result is confirmed by the fact that RNase A does not digest polyI as there was no significant change in its absorbance at 665 nm in the presence of methylene blue over a time period of 60 minutes.

### EXAMPLE 9

### Inhibition of RNase A Digestion of PolyA by the Addition of PolyG or PolyI

This example describes the inhibition of the degradation of polyA in the presence of polyG or polyI. RNase activity was measured by the absorption at 665 nm of a solution containing PolyA and methylene blue as described in Example 8. Reactions contained 4 μg/ml polyA solution and PolyI (Sigma, P4154) or PolyG (Sigma, P4404), either 2 μg or 4 μg in a final volume of 25 μl and were set up in duplicate. Ten microliters of 2 mg/ml RNase A (4 μg/ml final concentration) was added to each tube at time zero. Methylene blue was added at a concentration of 4 μg/ml and absorbance time points were taken at 60 and 100 minutes. Both PolyI and PolyG were found to inhibit the digestion of PolyA by RNase A as evidenced by the smaller change in absorbance upon the addition of RNase A. The results are shown below in Table 8 and demonstrate that the presence of 2 μg or 4 μg of polyI or polyG significantly lowered the RNase activity of RNase A on a polyA template.

TABLE 8
OD665 In The Presence of Methylene Blue and RNA Polymers

| Incubation | | | | | |
|---|---|---|---|---|---|
| Time | Polymer | | | | |
| (min) | None | 2 μg polyI | 4 μg polyI | 2 μg polyG | 4 μg polyG |
| 60 | 0.0785 | 0.0101 | −0.0153 | 0.0216 | 0.0076 |
| 100 | 0.116 | 0.04835 | −0.0112 | 0.04845 | 0.02905 |

### EXAMPLE 10

### Effect of the Timing of Addition of PolyI or PolyG on Inhibition of RNase Activity

Earlier experiments had demonstrated that the inhibition of RNase A by polyG polymer is not strictly competetive since preincubation of the enzyme with the polymer, followed by dilution, did not remove the inhibition. That result is further demonstrated in this example.

Fifty microliters of a 2mg/ml solution of RNaseA enzyme was combined with:

solution a: 50 μl 1 mg/ml polyG polymer (Sigma Corp.)

solution b: 50 μl nanopure water

solution c: 50 μl 1 mg/ml polyI polymer (Sigma Corp.)

A polyA stock solution of 20 μg/ml solution of PolyA in 100 mM NaOAc (pH 6.0) was prepared and contained methylene blue to track the OD655. The following six reactions were assembled in duplicate:

1. 5 ml of 100 mM NaOAc pH6.0 at time=0, 20 μl solution b added (blank)

2. 5 ml of polyA stock solution, at time=0, 20 μl solution b added (no polymer)

3. 5 ml of polyA stock solution, at time=0, 20 μl solution a added (polyG)

4. 5 ml of polyA stock solution, at time=0, 20 μl solution c added (polyI)

5. 5 ml of polyA stock solution with 2μg/ml poly G, at time=0, 20 μl solution b added

6. 5 ml of polyA stock solution with 2μg/ml polyI, at time =0, 20μp solution b added.

The six reactions were sampled at 60 minutes and 120 minutes and absorbance measured at OD655nm. The average OD655 results are listed in Table 9 below:

TABLE 9
OD655 In The Presence of Methylene Blue and RNA Polymers

| Reaction | time = 0 min | time = 60 min | time = 120 min |
|---|---|---|---|
| 1. blank | 1.8668 | 1.8723 | 1.87645 |
| 2. no inhibitor | 0.9909 | 1.13245 | 1.429 |
| 3. PolyG - early | 0.96095 | 0.9961 | 1.20595 |
| 4. PolyI - early | 0.91855 | 0.942 | 1.171 |
| 5. PolyG - late | 0.9645 | 0.9705 | 1.1414 |
| 6. PolyI - late | 0.9228 | 1.0332 | 1.342 |

The data demonstrate that the timing of addition of RNA polymers effects the digestion of polyA. The effect is particularly pronounced polyI polymer. PolyG and polyI with pre-incubation, show about the same inhibition against 20 μg of polyA as with 5 μg and no pre-incubation.

### EXAMPLE 11

### Inhibition of RNAses with Polymeric RNA Resin

This example describes the use of RNA polymers attached to resins to remove RNases from solutions. PolyG, polyI, and a combination of polyG and polyI were each coupled to cyanogen bromide activated agarose beads according to standard procedures well known in the art. The polymeric resins were purified and 100 μl of resin was added to individual 1.5 mL tubes. In additon, 100 μl of G-25 sephadex was added to a 4th tube as a control. The supernatant was removed from each tube and 1 ml of a solution containing 10 μg/ml RNAse A and 100 μg/ml BSA in 50 mM NaOAc pH 6.0 was added to each tube. The tubes were gently mixed for 15 minutes. The resin was allowed to settle in the tubes and 30 μl samples were taken from each tube. The samples were added to 1 ml of a solution of toluidine blue and RNA solution. RNase activity was measured by a decrease in absorbance at 650 nm. Alternatively, RNase activity was measured by spotting samples on RNase detection plates containing toluidine blue and RNA.

One milliliter of the polyG resin was added to a 2 ml column and the resin allowed to settle for 30 minutes. A 3ml solution was made containing 100 μg RNase B, 100 μg/ml BSA in 50 mM NaOAc, pH 6.0. The solution was applied to the polyG column and 0.5 ml fractions collected until all 3 mls passed through. The column was washed with 2 mls 50 mM NaOAc. Then 2 mls of 2 M NaCl was added to the column, still collecting fractions of flowthrough. After all the fractions were collected, 3 μl of each fraction was spotted on a RNase detection plate. As a control, a 3 μl spot of the original RNase B/BSA solution was spotted onto the plate. The control took 30 minutes for digestion to occur. The first fraction collected after the NaCl wash showed RNA digestion after 30 minutes also. No other fractions show RNA digestion after 45 minutes. No digestion was seen after 90 minutes in fractions preceeding the salt wash.

Likewise, RNase B was passed through a column of polyI resin and through a column of polyI+polyG resin. In both cases, RNA digestions was seen only in fractions directly after the NaCl wash.

### EXAMPLE 12

### Kinetics of Inhibition of RNase Enzymes by Polymeric RNAs

A spectrophotometric activity assay was used to measure RNase A activity. The hydrolysis of polyC (Sigma) by RNase A was measured by an increase in absorbance at 255 nm (Delcardayre et al., Protein Engineering, 8:261 [1995]). The assay was performed in a buffer containing 100 mM MES; 100 mM NaCl at pH 6.0. To observe linear kinetics, RNase levels can be between 1-10 ng and RNA concentrations (polyC was used as substrate in this example) can be between 5 μg/ml and 100 μg/ml (6.6-132 nm). Reaction components were mixed in a clean quartz cuvette and readings were taken every 10 seconds for 2 minutes.

Data was plotted as time vs. absorbance and the velocity was calculated from the linear portion of the curve.

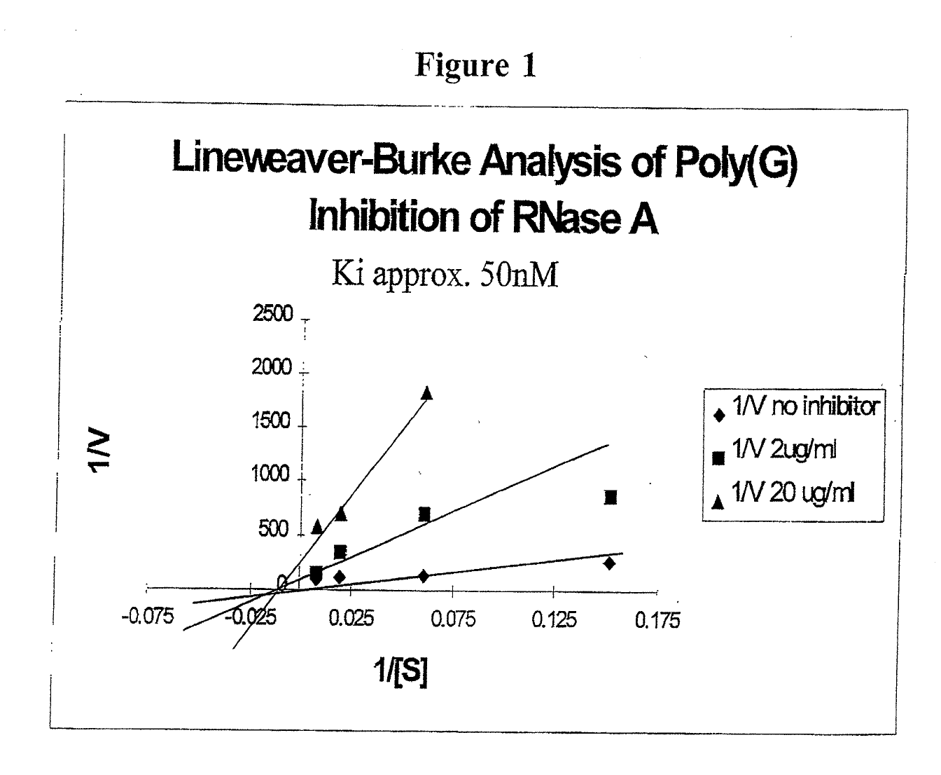

Inhibition constants were calculated using the assay conditions described above. PolyC substrate concentrations of 6.6 nM; 9.8 nM; 16.5 nM; 52.6 nM; and 132 nM were used. PolyG concentrations of 2 μg/ml and 20 μg/ml in nanopure water were used. Each reaction contained 7.5 ng of RNase A. A lineweaver-Burke analysis of the data (FIG. 1) indicated that the inhibition by polyG is non-competitive. Inhibition constants (Kᵢ) were calculated for both levels of polyG inhibitor and found to be 10.01 nM for 2 μg/ml PolyG and 87.3 nM for 20 μg/ml polyG.

Additional kinetic experiments were performed under different reaction conditions in order to determine the effects of pH and spermidine on inhibition of RNase A by PolyG. Reactions were performed in 40 mM Tris-HCl (pH 7.9); 6 mM MgCl₂ and 10 mM NaCl. One set of reactions contained 2 mM spermidine. A polyC substrate concentration of 16.5 nM and polyG concentrations of 2 μg/ml; 20 μg/ml; 50 μg/ml; and 100 μg/ml were used. RNAse A was added at 1 μg/ml (10 ng). A Dixon analysis was used to calculate inhibition constants. A Kᵢ of 10 nM in the absence of spermidine and 40 nM in the presence of spermidine was obtained. The increased pH of the reaction conditions did not change the Kᵢ for inhibition of RNase A by polyG.

The inhibition of RNAse ONE Ribonuclease (Promega) by polyG and 3-fluoro-4,6-dinitrophenyl (FNDP) modified polyG was measured. FNDP is a strong hydrophobic group that is coupled to the 2′-OH groups of the polyG backbone. Inhibition by polyG reversible. Depending on equilibrium conditions, the enzyme can become “unbound” from the inhibitor and regain activity. FDNP-polyG is an irreversible or “suicide” inhibitor. The inhibitor binds in the active site and forms a covalent bond with the enzyme. This happens when the 5-F atom in FDNP is displaced by a strong nucleophile. The formation of the covalent linkage permanently inactivates the enzyme. This experiment was designed to determine if FNDP-PolyG is a better inhibitor of RNase ONE due to the irreversible nature of the inhibition. Reactions were performed in PBS at pH 6.8. Five micrograms of RNase ONE Ribonuclease was incubated with either 20 μg or 80 μg of polyG for 1 hour at room temperature. At time points t=0, 30 min., 60 min., aliquots were removed and diluted 1:100 in 1× PBS. Then 10 μl of each dilution was added to 132 nM polyC substrate and the RNase activity was measured in the standard assay described herein above. The rate of substrate hydrolysis by the RNase ONE Ribonuclease was inhibited to an equal extent by both of the polyG substrates, indicating that the modification is not required for inhibition. Complete inhibition was observed at t=30 min. The nature of the assay and the order of addition of reaction components suggested the inhibition was non-competitive.

### EXAMPLE 13

### Binding Stoichiometry of RNase ONE Ribonuclease with PolyG

This examples describes the determination of the binding ratio of RNase ONE Ribonuclease to RNA polymers. The binding ratio of RNase ONE Ribonuclease to inhibitor RNA polymers was determined using the method descibed by Rahman et al. (Anal. Chem., 68:134 [1996]). Briefly, quenching of RNase ONE Ribonuclease fluorescence upon binding to PolyG was used to determine the binding stoichiometry. The total concentration of inhibitor+enzyme was kept constant and their molar ratios of enzyme to inhibitor were varied. The molar ratio at which the maximum change in fluorescence intensity occured was used to determine the molar ratio of binding.

Reactions were performed in PBS (pH 7.6) RNase ONE Ribonuclease concentrations varied from 0.25 to 1.0 μM and polyG concentrations varied from 0 to 0.75 μM. Enzyme and inhibitor were combined at various ratios in a cuvette and the fluorescence intensity was measured on a spectrofluorometer. Excitation of the complex was 295 nm and the fluorescence emission was monitored from 300 nm to 460 mn. Maximum fluorescence change occured at a molar ratio of 0.75 ([RNaseONE]/(RNaseONE]+[PolyG])), indicating that one molecule of PolyG binds three RNase ONE ribonuclease molecules.

### EXAMPLE 14

### Inhibition of Angiogenin RNase

This example demonstrates that both polyG and RNASIN Ribonuclease Inhibitor completely inhibited the RNase activity of angiogenin in vitro.

Angiogenin (R&D Systems) was dissolved in nuclease-free water to a final concentration of 0.1 mg/ml and then stored at −70° C. PolyG (Sigma P-4404) was dissolved in nuclease-free water to a final concentration of 2 mg/ml and used fresh. The following five reactions were assembled, the angiogenin was added last.:

TABLE 10
Reaction Conditions For Example 14

| Component | Rxn 1 | Rxn 2 | Rxn 3 | Rxn 4 | Rxn 5 |
|---|---|---|---|---|---|
| 1.2 Kb RNA | 2 μl | 2 μl | 2 μl | 2 μl | 2 μl |
| (0.5 μg/μl) | | | | | |
| 10× PBS, pH 6.8 | 2 μl | 2 μl | 2 μl | 2 μl | 2 μl |
| 0.1 mg/ml | 0 | 1 μl | 1 μl | 1 μ1 | 0 |
| Angiogenin | | | | | |
| 2 mg/ml polyG | 0 | 0 | 1 μl | 2 μl | 0 |
| 40 U/μl RNASIN | 0 | 0 | 0 | 0 | 3 μl |
| nuclease-free | 16 μl | 15 μl | 14 μl | 13 μl | 12 μl |
| water | | | | | |

The reactions were then incubated at 37° C. for 45 minutes and then run into an agarose gel and visualized by ethidium bromide staining. The gel showed that 100 μg and 200 μg PolyG and 120 U RNASIN ribonuclease inhibitor completely inhibited the RNAse activity of 100 μg angiogenin. While the RNA sample from reaction 2 was completely degraded, the RNA sample from the other reactions was intact.

Reactions similar to reaction number 3 above, but with amounts of polyG added to the reaction modified to 1 μg, 0.5 μg, 0.2 μg, 0.1 μg, and 0.02 μg, were assembled, incubated, and analyzed on a gel as described above. The gel showed that the lowest amount of polyG (0.02 μg) significantly inhibited the RNase activity of 0.1 μg in the presence of 1 μg RNA.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, chemistry, biochemistry, or related fields are intended to be within the scope of the following claims.

We claim:

1. A method for reducing the activity of an RNase, comprising:
a) providing
i) a preparation comprising at least one RNA polymer;
ii) a sample containing an RNase; and
b) mixing said preparation with said sample under conditions such that the activity of said RNA binding enzyme is diminished relative to the activity of said RNase in the absence of said RNA polymer.

2. The method of claim 1, wherein the activity of said RNase is diminished at least 25% relative to the activity of said RNase in the absence of said RNA polymer.

3. The method of claim 1, wherein the activity of said RNase is diminished at least 50% relative to the activity of said RNase in the absence of said RNA polymer.

4. The method of claim 1, wherein the activity of said RNase is diminished at least 75% relative to the activity of said RNase in the absence of said RNA polymer.

5. The method of claim 1, wherein the activity of said RNase is diminished at least 90% relative to the activity of said RNase in the absence of said RNA polymer.

6. The method of claim 1, wherein said one or more RNA polymers are selected from the group consisting of. polyA:polyU; polyC:polyG; polyC:polyI; polyI; polyC; polyA; polyG; poly(GU); poly(CU); poly(GI) and poly(CI).

7. The method of claim 1, wherein said one or more RNA polymers are affixed to a solid support.

8. The method of claim 7, wherein said solid support is a resin.

9. The method of claim 7, wherein said solid support comprises a plastic surface.

10. The method of claim 1, wherein said RNAse is selected from the group consisting of: RNase A, RNase H, RNase One, RNase B, RNase T₁, RNase T₂, RNase S, RNase from chicken liver, and RNase from Aspergillus clavatus.

11. The method of claim 1, wherein said preparation further comprises a ribonuclease inhibitor.

12. The method of claim 1, wherein said RNase is in a cell.

13. The method of claim 12, wherein said cell is a tumor cell.

14. The method of claim 1, wherein said RNase comprises angiogenin.

15. The method of claim 11, wherein said ribonuclease inhibitor is RNASIN.

16. A method of selling an RNase inhibitor to a customer, comprising providing a kit containing at least one RNA polymer to a customer for the purpose of inhibiting an RNase.

17. The method of claim 16, wherein said one or more RNA polymers are selected from the group consisting of: polyA:polyU; polyC:polyG; polyC:polyI; polyI; polyC; polyG; polyA; poly(GU); poly(CU); poly(GI) and poly(CI).

18. The method of claim 16, wherein said kit further comprises RNASIN RNase inhibitor.

19. The method of claim 16, wherein said kit further comprises a delivery system.

20. The method of claim 19, wherein said delivery system comprises a solid support.

21. The method of claim 20, wherein said solid support is a resin.

22. The method of claim 20, wherein said one or more RNA polymers are affixed to said solid support.

23. The method of claim 16, wherein said RNA polymer provided to said customer was obtained from RNA polymer batch, wherein prior to said providing said kit to said customer, at least a portion of said RNA polymer batch is tested in an RNase inhibition assay.
